(19) United States
(12) Patent Application Publication
(10) Pub. No.: US 2005/0238675 A1
(43) Pub. Date: Oct. 27, 2005

(54) Water-soluble formulations of fat soluble vitamins and pharmaceutical agents and their applications

(76) Inventors: Wenjie Li, Ladera Ranch, CA (US); Amy Nguyen, Newport Beach, CA (US); Edward Alosio, Coto De Caza, CA (US); Bricini Faith Dema-Ala, Aliso Viejo, CA (US)

Correspondence Address: GABOR L. SZEKERES, 8141 E. KAI SER BLVD., SUITE 112, ANAHEIM HILLS, CA, 92808, US

(21) Appl. No.: 11/109,548

(22) Filed: Apr. 19, 2005

Related U.S. Application Data
(60) Provisional application No. 60/565,363, filed on Apr. 26, 2004.

(51) Int. Cl. A61K 31/57; A61K 31/355; A61K 31/22

(52) U.S. Cl. 424/400; 514/546; 514/458; 514/560; 514/171

(57) ABSTRACT

Aqueous liquid or gel formulations of fat soluble vitamins, essential nutrients and other pharmaceutical agents have enhanced concentration of the active components relative to known compositions and therefore have enhanced bioavailability. The aqueous solutions or gels form a free flowing powder when they are absorbed on a suitable pharmaceutically acceptable solid carrier, such as silicon dioxide, maltodextrin, magnesium oxide, aluminum hydroxide, magnesium trisilicate, starch or sugars, or encapsulated by polymers such as gelatin, pectin, chitosan and the like.

### FIELD OF THE INVENTION

The present invention is directed to water-soluble formulations of fat soluble vitamins, essential nutrients and other pharmaceutical agents. The present invention is also directed to free-flowing solid formulations of fat soluble vitamins, essential nutrients and other pharmaceutical agents, which per se are poorly soluble in water, and where the formulation nevertheless provides improved bio-availability of the vitamin, essential nutrient or other pharmaceutical agent.

### BRIEF DESCRIPTION OF BACKGROUND ART

Microemulsions and micellized formulations have been employed in the prior art to make fat soluble and/or poorly water soluble vitamins, essential nutrients and pharmaceutical agents, such as drugs, available for human consumption and/or to increase their bioavailability after ingestion.

U.S. Pat. No. 4,572,915 discloses a process of micellizing fat-soluble vitamins, essential oils and other fat-soluble agents for liquid preparations in nutritional supplements and cosmetics. Clinical trials with micellized vitamin A and E showed 3-5 times more absorption of these vitamins than those in edible oils. Unlike microemulsions, micellized fat-soluble vitamins can be added to water and result in transparent solutions.

U.S. Pat. Nos. 6,143,321; 6,110,490; 6,309,665; 6,312,704; 5,444,041; 5,993,858; 5,972,911; 5,989,583; 6,337,087; 6,103,259; 6,146,825; 6,337,087; 6,231,882; 6,130,209; 6,120,794; 6,017,545; 6,013,665; 6,248,360; 6,054,136; 6,346,273; 6,027,747; 6,280,770 and 6,248,363 also are of interest as background to the present invention because they relate to microemulsions and to self-microemulsifying drug delivery systems (SMEDDS), to increase the bio-availability of poorly water-soluble drugs A desirable feature of SMEDDS is their ability to form microemulsions when exposed to gastrointestinal fluids. Reviewing the previous arts, the big problem to prevent their application is low drug load in the formulation, typically carrying less then 1% of the active component or components. Because of low drug load, it is only practical for high potency drugs/nutrients. In supplemental applications, these fat-soluble nutrients are often combined with other nutrients, flavors and sweeteners to form complex products. It is often challenge to formulate a market-acceptable product combining all these nutrients together in liquid form. Therefore, there is a need to develop a process to increase drug/nutrient load for any meaningful application.

### SUMMARY OF THE INVENTION

The present invention provides liquid or gel formulations for fat soluble vitamins, fat soluble essential nutrients and other pharmaceutical agents.

In accordance with the present invention a liquid or gel composition is obtained that contains the following ingredients or components.
(1) 5 to 60 per cent by weight of a pharmaceutically acceptable surfactant, preferably 10 to 40% by weight of the pharmaceutically acceptable surfactant;
(2) 1-50 per cent by weight of water, preferably 3 to 50 per cent by weight of deionized water:
(3) 0 to 20 per cent by weight of an unsaturated fatty acid ester, preferably 3 to 10 per cent by weight of the unsaturated fatty acid ester;
(4) 0 to 50 per cent by weight of a water miscible pharmaceutically acceptable polyol, preferably 4 to 40 per cent by weight of the pharmaceutically acceptable polyol;
(5) 0 to 10 per cent by weight of a pharmaceutically acceptable phospholipid, preferably 1 to 5 per cent by weight of the phospoholipid, and
(6) 1 to 40 per cent by weight of one more lipophilic or fat soluble vitamin, essential nutrient, pharmaceutical agent or drug, preferably 1 to 30 per cent by weight of the lipophilic or fat soluble vitamin, essential nutrient, pharmaceutical agent or drug.

### DESCRIPTION OF THE INVENTION

The present invention provides clear liquids or gels of lipophilic or fat soluble vitamins, essential nutrients, or other pharmaceutical agents of good or improved bioavailability. The present invention also provides solid formulations of lipophilic or fat soluble vitamins, essential nutrients, or other pharmaceutical agents of good or improved bioavailability.

The formulations of the present invention provide a significant improvement or advantage in terms of bio-availability of the lipophilic or fat soluble vitamins, essential nutrients, or other pharmaceutical agents which have relatively low aqueous solubility and which in prior art liquid, gel or solid formulations have lesser bio-availability.

A principal ingredient or component of the formulations of the present invention is comprised by one or more lipophilic or fat soluble vitamins, essential nutrients, or other pharmaceutical agent, which in the liquid formulation is present in 1 to 40 per cent by weight, and preferably in 1 to 30 per cent by weight.

Examples, without intending any limitation, of the lipophilic or fat soluble vitamins, essential nutrients, or other pharmaceutical agents are: fat soluble derivatives of Vitamin A, such as Vitamin A palmitate, beta carotene, fat soluble derivatives of tocopherol, such as d-alpha tocopheryl acetate, co-enzyme Q10 (also known as ubidecarenone), fish oils and drugs such as simvastitin. In accordance with the present invention more than one of the above-exemplified or other fat soluble vitamins, essential nutrients or other pharmaceutical agents may also be provided in a single formulation of improved bioavailability.

Another important or principal component of the formulations of the present invention is a pharmaceutically acceptable surfactant or emulsifying agent, examples of which are polyoxyethylene sorbitan fatty acid esters, polyoxyethylene alkyl ethers, polyoxyethylene castor oil derivatives, polyoxyethylene stearates, and saturated polyglycolized glycerides. These pharmaceutically acceptable surfactants are well known in the art and are available from commercial sources.

Specific examples of the surfactants that are used to prepare the preferred embodiments or examples of the present invention are: POE(20) sorbitan monooleate (available under the commercial name Polysorbate 80 Glycosperse 0-20); polyoxyl 4-lauryl ether (available under the commercial name Brij 30); polyoxyl 35 castor oil (available under the commercial name as Cremophor EL); lauroyl macrogol-32 glycerides (available under the commercial name as Gelucire 44/14); polyoxyl 50 stearate (available under the commercial name Myrj 53); diethylene glycol monoethyl ether (available under the commercial name Transcutol P). The pharmaceutically acceptable surfactant or emulsifying agent is present in the liquid or gel formulations of the present invention in the ratio of 5 to 60 per cent by weight, preferably in the ratio of 10 to 40 per cent by weight.

A function of the surfactant or emulsifying agent is to stabilize in conjunction with the other components and likely in micelles, and thereby solubilize, again in conjunction with the other components, the lipophilic or fat soluble essential nutrient, vitamin or other pharmaceutical agent or a plurality of the lipophilic or fat soluble essential nutrients, vitamins or other pharmaceutical agents. As noted above, the lipophilic or fat soluble essential nutrient, vitamin or other pharmaceutical agent or a plurality of the lipophilic or fat soluble essential nutrients, vitamins or other pharmaceutical agents included in the formulation are likely to have poor aqueous solubility, and without the solubilization that occurs through micellization, only a significantly lesser amount of the drug could be dissolved in the amount of water used in the formulation, and the increased bio-availability could not be achieved.

The surfactant or emulsifying agent used in the formulation can be a single product, or a combination of two or more of the products or components identified above. Generally speaking, where more than one chemical compound or substance of a certain general category (such as surfactant, unsaturated fatty acid ester, polyol, or phospholipid, preservative or flavoring agent etc.) can be utilized in the present invention, then instead of a single such compound or substance a combination of substances falling within the same general category can also be used.

Still another ingredient or component of the liquid and gel formulations of the present invention is water that is present in the range of 1 to 50 per cent by weight. Preferably the water is deionized, distilled or otherwise purified water and is present in the range of 3 to 50 per cent by weight.

Yet another ingredient or component of the formulations of the present invention is an unsaturated fatty acid ester which is present in the range of 0 to 20 per cent by weight, preferably 3 to 10 per cent by weight. Actually, the unsaturated fatty acid ester is a desired but nevertheless only optional component of the formulations of the invention, and that is the reason why its possible range, broadly speaking, begins at zero (0) per cent. The ester of the unsaturated fatty acid, such as ethyl linoleate, acts as a solubilizing agent. Other examples of suitable unsaturated fatty acids are palmitoleic acid, oleic acid, linoleic acid, which can be present in the composition individually or in combination.

A further ingredient component of the formulations of the present invention is a water miscible and pharmaceutically acceptable polyol, the preferred examples of which are glycerol and propylene glycol. Examples of other suitable water miscible and pharmaceutically acceptable polyols are diethylene glycol, diethylene glycol monoethyl ether (available under the commercial name Transcutol P) and polyethylene glycol. The water miscible, pharmaceutically acceptable polyol acts as an emulsifying or solubilizing agent and also increases the viscosity of the liquid or gel formulations which are first obtained in accordance with the present invention. However, the water miscible and pharmaceutically acceptable polyol is not absolutely essential for preparing the formulations of the present invention, and for this reason, broadly speaking, its range in the liquid or gel formulations of the invention is indicated as zero (0) to 50 per cent by weight. Nevertheless, the inclusion of a water miscible and pharmaceutically acceptable polyol or polyols in the formulations is preferred in the range of 4 to 40 per cent by weight. Glycerol and/or propylene glycol is/are present in all liquid or gel embodiments of the invention described below. In several embodiments it may be advantageous to include both glycerol and propylene glycol. A still-further ingredient or component of the formulations of the present invention is comprised of phospholipids. However, the phospholipid is only an optional but nevertheless preferred ingredient. For this reason, in the broadest sense, the range of phospholipid in the formulations of the present invention is zero (0) to 10 percent by weight, preferably 1 to 10 percent by weight and even more preferably 1 to 5 per cent by weight. The function of the phospholipids is also to solubilize the drug or pharmaceutical agent. A preferred example of the pharmaceutically acceptable phospholipids included in the formulations of the present invention is lecithin. Other examples of phospholipids suitable for incorporation in the present invention are phosphatidylethanolamine, phosphatidylserine, phosphatidylinositol. The phospholipid, such as lecithin, can be added in aqueous solution, in which case the water of this solution provides some or all of the water utilized to dissolve and solubilize the above listed components to obtain either a gel or a liquid solution.

Optional ingredients, such as preservatives (for example sodium benzoate, potassium sorbate or parabene) and flavoring agents, sweeteners (such as xylitol, sorbital or the like) and still other ingredients well known in the art for use in liquid and gel formulations may be included in relatively small quantities (for example 0.2 to 3 per cent by weight) in the formulations of the present invention.

The above-noted components are thoroughly admixed in accordance to provide a clear liquid or clear gel. Generally speaking, the process of admixing requires heating to elevated temperature a combination of one or more components and slowly adding under vigorous stirring the other ingredients which may also be at elevated temperature. Those skilled in the art will readily understand that the nature and consistency of the formulations obtained in this manner (whether it is a liquid or gel, and the consistency of the gel) depend on the nature and amounts of the several components used. It should also be understood in connection with the herein listed ranges of percentages of the components, that it is not contemplated within the scope of the invention to have all or most of the ingredients present in their respective maximum listed range in any given composition, as such a composition would be incapable of existence for having more than 100% of the sum of its components. Rather, it is contemplated that when one or more ingredients are in their maximum range, then the ratios of other components are in less than their maximum range, so that the sum total of all components (listed or not listed above) is 100%.

In accordance with another aspect of the present invention the gel or liquid formulations of the present invention are absorbed on a suitable pharmaceutically acceptable solid carrier, such as silicon dioxide, maltodextrin, magnesium oxide, aluminum hydroxide, magnesium trisilicate, starch or sugars, such as fructose, or encapsulated by polymers such as gelatin, pectin, chitosan and others. Among these solid carriers maltodextrin and silicon dioxide, particularly colloidal silicon dioxide, are presently preferred. These carriers per se well known in the art, and need not be described here further. The gel or liquid formulations can be absorbed by the solid carrier either by granulation or by spray drying. Both the granulation and spray drying processes are well known in the art, and need not be described here further. The gel or liquid formulation can be further encapsulated by coacervation or interfacial polymerization. The liquid or gel formulations absorbed in this manner on the solid carrier or encapsulated become free-flowing powders that are suitable as such for being formed into tablets or capsules. However, other pharmaceutically acceptable excipients can also be added to the free-flowing powder obtained in the above-described manner to make tablets or capsules or other solid forms suitable for practical oral administration. In addition, coloring agents, flavoring agents or preservatives and other pharmaceutically acceptable substances that are normally or occasionally included in tablets or capsules in addition to the fat soluble vitamins, essential nutrients or other pharmaceutical agents, may also be included in the tablets or capsules. Such non-active components may, also be added to the formulation while it is a liquid or gel, or before the components are admixed to form a liquid or gel.

Generally speaking, the free flowing powder obtained from the gel or liquid includes 20 to 80 per cent by weight of the gel or liquid and 20 to 80 per cent by weight of the solid carrier. More preferably, the free flowing powder obtained from the gel or liquid includes 50 to 80 per cent by weight of the gel or liquid and 20 to 50 per cent by weight of the solid carrier. Tablets or capsules made by utilizing the free flowing powder may contain the same percentages as the free flowing powders or may be further diluted by other excipients, such as microcrystalline cellulose, dicalcium phosphate, stearic acid and magnesium stearate.

The fat soluble vitamins, essential nutrients or other pharmaceutical agents have improved bio-availability to mammals, including humans, when administered in the formulations of the present invention.

### SPECIFIC EXAMPLES

In the below-given examples all percentages are by weight.

The term QS in these specific examples means that sufficient 5% aqueous lecithin ( or water) solution is added to the composition to make 100 per cent. The lecithin solution in this example is 5 percent weight by weight. Thus, if one were to make a 100 grams total of the formulation, for example, of Example 1, then 38.78 grams of 5% aqueous solution would be combined with the other components. 38.78 grams of 5% aqueous lecithin solution contains 1.94 g lecithin (phospholipid) and 36.84 grams of water.

### Example 1

| | |
|---|---|
| Polysorbate 80 Glycosperse O-20 | 30% |
| Beta-Carotene 30% oil | 6.22% |
| Glycerine | 20% |
| Ethyl Linoleate | 5% |
| 5% Lecithin solution | QS |

Procedure: Into a clean beaker, weigh in Polysorbate 80 and Beta-Carotene 30% oil. With mixer on, heat to 160-180 ° C. until clear and homogeneous. Cool to 90° C. and add Glycerine and Ethyl Linoleate. Maintain temperature @ 85-90° C. and mix until clear homogeneous (Part I). In a separate beaker, make 5% Lecithin aqueous solution at 60° C. Start cooling Part I and gradually add 5% Lecithin solution into Part I to final batch weight. Mix until clear and homogeneous. The cooled gel is clear and homogeneous and miscible with water to form a clear solution.

Solubility Measurement: For comparison purpose, the solubility of beta-carotene was measured both in edible oil and in the micellized product of Example 1 described above. In these measurements a suitable amount of sample is mixed with 10 ml of DI water for at least 10 minutes. The solutions are filtered through 0.45 microns syringe filter. The beta-carotene contained in the sample after this filtration is measured by UV-Vis spectrometry. No detectable amount of beta-carotene found in the aqueous sample where the beta-carotene was admixed in oil. 100% of the originally admixed beta-carotene is measured to be present in the micellized filtered sample of Example 1.

### Example 2

| | |
|---|---|
| Polysorbate 80 Glycosperse O-20 | 25% |
| Vitamin A Palmitate 1.7 MM IU/g | 5% |
| Glycerine | 15% |
| 5% lecithin aqueous solution | 45% |
| DI Water | QS |

Procedure: Into a clean beaker, weigh in Polysorbate 80 and glycerine and heat to 50-90° C., with mixer on, Gradually add other items. Slowly add 5% lecithin aqueous solution until clear and use water to adjust to final batch weight. Mix until clear and homogeneous. The cooled solution is clear and homogeneous and miscible with water to form a clear solution.

The above procedure of preparation applies to Examples 2 to 5

### Example 3

| | |
|---|---|
| Polysorbate 80 Glycosperse O-20 | 40% |
| d-alpha Tocopheryl Acetate 1.1 M IU/g | 40% |
| Propylene Glycol | 10% |
| Ethyl Linoleate | 3.0% |
| 5% lecithin aqueous solution | 6% |
| DI Water | QS |

Solubility Measurement: Assay is done by high pressure liquid chromatography (HPLC). When Vitamin E acetate is dispersed in oil and thereafter the dispersion/solution is mixed with water, the mixture filtered on a 0.45 micron filter and thereafter assayed by HPLC, there is no vitamin E detected in the aqueous part of the filtered sample. The HPLC measurement results in recovery of 95.9% of the originally added vitamin E acetate in the filtered micellized sample of Example 3.

### Example 4

| | |
|---|---|
| Polysorbate 80 Glycosperse O-20 | 25% |
| Co-enzyme Q10 | 15% |
| Sorbital | 6.0% |
| Glycerine | 6% |
| Ethyl Linoleate | 5% |
| d-Alpha-tocopherol | 0.2% |
| 5% lecithin aqueous solution | 7.5% |
| DI Water | QS |

Solubility Measurement: Assay is done by HPLC. For Co-Q10 powder, there is no Co-Q10 detected in aqueous solution while there is 93.1% of the material recovered in micellized form.

### Example 5

| | |
|---|---|
| Polysorbate 80 Glycosperse O-20 | 57.5% |
| High EPA Fish Oil | 33.8% |
| d-alpha Tocopherol Acetate 1.1 M IU/g | 1.1% |
| Vitamin A Palmitate, 1 .7 MM IU/g | 0.18% |
| Propylene Glycol | 4% |
| 5% lecithin aqueous solution | QS |

### Example 6

| | |
|---|---|
| Polysorbate 80 Glycosperse O-20 | 35% |
| Propylene Glycol | 25% |
| Ethyl Linoleate | 8% |
| Simvastatin | 4% |
| 5% Lecithin aqueous solution | QS |

Procedure: Polysorbate is heated to 120° C. Slowly add simvastatin into the above solution with vigorous stirring until homogeneous and clear. Slowly and consecutively add other ingredients into the solution with stirring. Finally, aqueous lecithin solution is added to make 100% with vigorous stirring. The mixture is then cooled immediately in a cold-water bath. The cooled gel is clear and homogeneous and miscible with water to form a clear solution.

### Example 7

| | |
|---|---|
| Polyoxyl 4-lauryl ether (Brij 30) | 35% |
| Propylene Glycol | 25% |
| Ethyl Linoleate | 8% |
| Simvastatin | 4% |
| 5% Lecithin aqueous solution | QS |

Follow the procedure of Example 6.

### Example 8

| | |
|---|---|
| Lauroyl macrogol-32 glycerides | 35% |
| (Gelucire 44/14) | |
| Propylene Glycol | 25% |
| Ethyl Linoleate | 8% |
| Simvastatin | 4% |
| 5% Lecithin aqueous solution | QS |

### Example 9

| | |
|---|---|
| Polysorbate 80 Glycosperse O-20 | 35% |
| Propylene Glycol | 25% |
| Ethyl Linoleate | 8% |
| Progesterone | 4% |
| 5% Lecithin aqueous solution | QS |

Procedure for Examples 8, and 9: Into a clean beaker, weigh in Polysorbate 80 and heat to 50-80° C., with mixer on, Gradually add other items. Mix until clear before adding next item. Use 5% lecithin aqueous solution to adjust to final batch weight. Mix until clear and homogeneous. The cooled solution is clear and homogeneous and miscible with water to form a clear solution.

### Example 10

| | |
|---|---|
| Polysorbate 80 Glycosperse O-20 | 44% |
| Vitamin A Propionate | 26% |
| Propylene Glycol | 13.375% |
| Ethyl Linoleate | 5% |
| Ascorbyl Palmitate | 1.625% |
| d-alpha-tocopherol | 1.5% |
| Ubidecarenone | 1% |
| 5% Lecithin aqueous solution | 7.5% |

Procedure: Into a clean beaker, weigh in Polysorbate 80 and vitamin A propionate and heat to 50-80° C. with mixer on, Gradually add Co-Q10, propylene glycol, ethyl linoleate, Vitamin E and ascorbyl palmitate. Mix until clear before adding next item. Use 5% lecithin aqueous solution to adjust to final batch weight. Mix until clear and homogeneous. The cooled solution is clear and homogeneous and miscible with water to form a clear solution.

### Example 11

### Micellized Fat-Soluble Vitamins

| | |
|---|---|
| Polysorbate 80 Glycosperse O-20 | 40% |
| Beta-Carotene 30% oil | 6.22% |
| Vitamin A Palmitate | 1.8301% |
| d-alpha-tocopherol | 2.9487% |
| Glycerine | 20% |
| Ethyl Linoleate | 4% |
| 5% Lecithin solution | QS |

Procedure: Weigh half of polysorbate into a beaker and add beta carotene (30% oil). Heat the mixture on a hot plate to 160-180° C. to form a clear and homogeneous solution with constant stirring. In a separate beaker, weigh in the rest of polysorbate. Raise the temperature to 80-90° C. Gradually add vitamin A palmitate, d-alpha-tocopherol, glycerine and ethyl linoleate to the beaker with vigorous stirring. Cool the first beaker to 120-130° C. and combine the content in the second beaker into the first one. Start cooling. At 60° C., add 5% lecithin aqueous solution to the batch weight. The cooled product is clear, dark-red gel and miscible with water to form a clear solution.

### Example 12

### Micellized Multi-Vitamins

| Part I | |
|---|---|
| Polysorbate 80 Glycosperse O-20 | 5% |
| Beta-Carotene 30% oil | 1.25% |
| Part II | |
| Polysorbate 80 Glycosperse O-20 | 7.5% |
| Vitamin A Palmitate | 0.286% |
| d-alpha-tocopheryl acetate | 3.5233% |
| Glycerine | 12.5% |
| Vitamin D3 oil | 0.0319% |
| Part III | |
| DI Water | 60% |
| Potassium Sorbate | 0.2% |
| Potassium Benzoate | 0.1% |
| d-Biotin | 0.0267% |
| Niacinamide | 3.1021% |
| Pyrodoxine HCL | 0.4308% |
| Riboflavin-5-Phosphate | 0.4043% |
| Thiamine Mononitrate | 0.328% |
| Cyanocobalamin | 0.0012% |
| d-panthenol | 2.1296% |
| DI water | 3.1861% |

Procedure: Make Part I and Part II according the procedure described in Example 11. In a separate beaker, weigh in DI water and heat to 50-60° C. Gradually add each items in Part III with mixing and make sure each item is completely dissolved before adding another. Add the mixture of Part I and Part II into the beaker and mix thoroughly. Finally QS to batch weight with additional DI water. The solution is clear and homogeneous and miscible with water to form a clear solution.

### Example 13

45% of maltodextrin is granulated with 55% of Vitamin E acetate gel prepared in Example 3 to yield a uniform wet granulation. The granule is dried at approximately 60-70° C. to provide a free-flowing powder. The powder is soluble in water to produce a clear solution. Assay of the powder in the solution shows 100% of Vitamin E acetate dissolved.

### Example 14

Micellized Fish Oil Gel Cap: The solution prepared in Example 5 is encapsulated in soft gelcap. The gelcap is amber-brown and soluble in water to result in a clear solution.

### Example 15

Rejuvenated Cream with Micellized Co-Q10, Vitamin A and Vitamin E: A Micelle portion is made according to Example 10. This portion of 0.8% is added into typical cream base to result in a smooth and creamy cream. Apply the cream to skin and the cream is absorbed into skin with no greasy feel.

### Example 16

0.3% of the sample prepared in Example 11, along with suitable antibacterial agents, sweeteners and flavors, is used to make BetaCell Mouth Rinse for prevention of oral lesions. The product is a clear solution.

### Example 17

### Liquid Multi-Vitamin Multi-Mineral Supplement

2% of the sample prepared in Example 12, along with other minerals, sweeteners and flavors, is used to make liquid multi-vitamin and multi-mineral supplement. The product is clear without any separation over a one year of period.

1. A formulation comprising:
(a) 5 to 60 per cent by weight of a pharmaceutically acceptable surfactant;
(b) 1-50 per cent by weight of water;
(c) 1 to 40 per cent by weight of one more active component selected from the group consisting of lipophilic or fat soluble vitamins, essential nutrients, pharmaceutical agents and drugs.

2. A formulation in accordance with claim 1 additionally comprising 4 to 20 per cent by weight of an unsaturated fatty acid ester, and 10 to 50 per cent cent by weight of a water miscible pharmaceutically acceptable polyol.

3. A formulation in accordance with claim 2 additionally comprising 1 to 10 per cent by weight of a pharmaceutically acceptable phospholipid.

4. A formulation in accordance with claim 1 where the formulation is a clear liquid.

5. A formulation in accordance with claim 1 where the formulation is a clear gel.

6. A formulation in accordance with claim 2 where the formulation is a clear liquid.

7. A formulation in accordance with claim 2 where the formulation is a clear gel.

8. A formulation in accordance with claim 3 where the formulation is a clear liquid.

9. A formulation in accordance with claim 3 where the formulation is a clear gel.

10. A formulation in accordance with claim 3 where the active component is beta carotene.

11. A formulation in accordance with claim 3 where the active component is Vitamin A Palmitate.

12. A formulation in accordance with claim 2 where the active component is d-alpha tocopheryl acetate or d-alpha tocopherol.

13. A formulation in accordance with claim 2 where the active component is Coenzyme Q10.

14. A formulation in accordance with claim 3 where the active component is EFA fish oil.

15. A formulation in accordance with claim 3 where the active component is a drug.

16. A formulation in accordance with claim 2 containing a plurality of active components.

17. A formulation in accordance with claim 3 containing a plurality of active components.

18. A formulation in accordance with claim 3 where the active component is progesterone.

19. A formulation comprising a free flowing powder prepared by the process of admixing a formulation in accordance within claim 1 with a pharmaceutically acceptable solid carrier.

20. A formulation comprising a free flowing powder prepared by the process of admixing a formulation in accordance within claim 2 with a pharmaceutically acceptable solid carrier.

21. A formulation comprising a free flowing powder prepared by the process of admixing a formulation in accordance within claim 3 with a pharmaceutically acceptable solid carrier.

22. A formulation in accordance with claim 19 where the pharmaceutically acceptable solid carrier is selected from the group consisting of silicon dioxide, maltodextrin, magnesium oxide, aluminum hydroxide, magnesium trisilicate, starch and sugars.

23. A formulation in accordance with claim 20 where the pharmaceutically acceptable solid carrier is selected from the group consisting of silicon dioxide, maltodextrin, magnesium oxide, aluminum hydroxide, magnesium trisilicate, starch and sugars.

24. A formulation in accordance with claim 21 where the pharmaceutically acceptable solid carrier is selected from the group consisting of silicon dioxide, maltodextrin, magnesium oxide, aluminum hydroxide, magnesium trisilicate, starch and sugars.

25. A formulation comprising:
(a) 5 to 60 per cent by weight of a pharmaceutically acceptable surfactant;
(b) 3-50 per cent by weight of water;
(c) 3 to 10 per cent by weight of an unsaturated fatty acid ester;
(d) 4 to 40 per cent by weight of a water miscible pharmaceutically acceptable polyol, and
(e) 1 to 30 per cent by weight of one more active component selected from the group consisting of lipophilic or fat soluble vitamins, essential nutrients, pharmaceutical agents and drugs.

26. A formulation in accordance with claim 25 where the formulation additionally comprises 1 to 5 per cent by weight of a pharmaceutically acceptable phospholipid.

27. A formulation in accordance with claim 25 where the formulation is a clear liquid.

28. A formulation in accordance with claim 25 where the formulation is a clear gel.

29. A formulation in accordance with claim 26 where the formulation is a clear liquid.

30. A formulation in accordance with claim 26 where the formulation is a clear gel.

31. A formulation comprising a free flowing powder prepared by the process of admixing a formulation in accordance within claim 25 with a pharmaceutically acceptable solid carrier.

32. A formulation comprising a free flowing powder prepared by the process of admixinig a formulation in accordance within claim 26 with a pharmaceutically acceptable solid carrier.

33. A formulation in accordance with claim 31 where the pharmaceutically acceptable solid carrier is selected from the group consisting of silicon dioxide, maltodextrin, magnesium oxide, aluminum hydroxide, magnesium trisilicate, starch and sugars.

34. A formulation in accordance with claim 32 where the pharmaceutically acceptable solid carrier is selected from the group consisting of silicon dioxide, maltodextrin, magnesium oxide, aluminum hydroxide, magnesium trisilicate, starch and sugars.
